(19) United States
(12) Patent Application Publication
(10) Pub. No.: US 2012/0148543 A1
(43) Pub. Date: Jun. 14, 2012

(54) SYNTHETIC GRAFT

(76) Inventor: Che Connon, Reading (GB)

(21) Appl. No.: 13/321,603

(22) PCT Filed: May 21, 2010
(86) PCT No.: PCT/GB2010/001024

§ 371 Date: Feb. 8, 2012

(30) Foreign Application Priority Data
May 22, 2009 (GB) 0908927.7

(51) Int. Cl. A61K 35/44; C12Q 1/68; A61P 27/02; G01N 21/76; C12N 5/071; C12Q 1/02

(52) U.S. Cl. 424/93.7; 435/397; 435/29; 435/7.1; 435/6.12

(57) ABSTRACT

The present invention relates to the use of a plastically-compacted collagen gel as a substrate for the growth of corneal cells, particularly limbal corneal epithelial stem cells. Cells grown on such a substrate can be cultured to produce artificial ocular epithelia which can be used in ocular toxicity testing or for transplantation.

The present invention relates to the use of a plastically-compacted collagen gel as a substrate for the growth of corneal cells, particularly limbal corneal epithelial stem cells. Cells grown on such a substrate can be cultured to produce artificial ocular epithelia or artificial corneal tissue which can be used in ocular toxicity testing or for transplantation.

Prior to commercialization, new drugs and cosmetics must be tested in oculotoxicity tests such as the Draize rabbit eye irritancy test in order to establish the toxic potential of those new drugs/cosmetics. The eye is used in this regard because it presents the most commonly-exposed and chemically-sensitive extremity to our everyday environment. Thousands of rabbits are used every year in such tests and this method of testing drugs and cosmetics on rabbits eyes has changed little over the last 50 years. The Draize rabbit eye test has been criticised, however, not only on ethical grounds but also on scientific grounds because of major differences between rabbit and human eyes. However, no non-animal test is currently accepted as a substitute for the Draize test; imminent changes in European legislation are likely to increase the need for such a replacement.

The use of in vitro alternatives to animal models have previously been investigated using organ culture, human cell lines and human donor tissue, but the effectiveness of these models has been hampered by genetic instability, two dimensional tissue culture limitations (not modelling the epithelial barrier function), lack of normal growth and differentiation, inter-species genetic variation and limited availability. For these reasons, the need for a three-dimensional (3D) corneal model has lead recently to the development of two commercial epithelium models (SkinEthic Laboratories and EpiOcular, MatTek Corp) as in vitro alternatives for eye irritation tests. The SkinEthic model uses immortalized human corneal epithelial cells (Doucet, O. et al. Toxicol In Vitro, 2006. 20(4): p. 499-512), while the MatTek model uses normal keratinocytes (Van Goethem, F. et al. Toxicol. In Vitro, 2006. 20(1): p. 1-17). Although both of these models display a cornea-like epithelial structure, neither use a physiological substrate, nor do they model the important role that corneal stem cells play in maintaining the function of the corneal epithelium.

Attempts have been made to provide a substrate for the growth of corneal cells which mimics the physiological substrate provided by the cornea in vivo. A wide range of substrates has been tried including amniotic membrane, temperature-sensitive hydrogels, plasma polymer coated substrates and collagen, fibrin, and fibronectin/fibrin gels. In a comparison between amniotic membrane, collagen gels and collagen shields as carriers for harvested corneal stem cells, amniotic membrane was found to be the superior carrier (Schwab, I. R. Trans. Am. Opthalmol. Soc. 1999, 97: p. 891-986). Since that time, amniotic membrane has been used as the standard corneal cell substrate because it encourages proliferation, adhesion and differentiation of cells grown on it. It has also been shown to be an excellent substrate for the clinical expansion of corneal stem cells for ocular surface transplantation (e.g. Koizumi N et al., Invest. Ophthalmol. Vis. Sci. 2000; 41:2506-2513).

However, amniotic membrane shows significant inter- and intra-sample variation in structure and chemical composition (Hopkinson, A. et al. Invest. Ophthalmol. Vis. Sci., 2006. 47(10): p. 4316-4322) and is not routinely characterised before clinical use. Most importantly, amniotic membrane as a substrate lacks the scalability of an engineered polymer construct.

Attempts have therefore been made to fabricate corneal epithelial graft constructs ex vivo from expanded limbal stem cells on substrates other than amniotic membrane. A substrate suitable for in vitro oculotoxicity testing using corneal stem cells needs to have the following basic requirements: (i) to sustain stem cell expansion and (ii) to provide a solid support for cell stratification. It is one object of the invention therefore to provide new types of substrates which offer similar tissue engineering capabilities to amniotic membrane but are more accessible and more easily standardised.

In one aspect, the invention provides the use of a plastically-compacted collagen gel as a substrate for the growth of corneal cells.

An uncompacted collagen gel comprises a matrix of collagen fibrils which form a continuous scaffold around an interstitial liquid. For example, dissolved collagen may be induced to polymerise/aggregate by the addition of dilute alkali to form a gelled network of cross-linked collagen fibrils. The gelled network of fibrils supports the original volume of the dissolved collagen fibres, retaining the interstitial liquid. General methods for the production of such collagen gels are well known in the art (e.g. WO2006/003442, WO2007/060459 and WO2009/004351).

As used herein, the term “plastically-compacted collagen gel” refers to a collagen gel whose original volume has been reduced by an external compacting/dehydrating treatment, wherein a portion of or the majority of the original interstitial liquid has been removed from the gel, and wherein the collagen gel has retained its new (reduced) volume after the removal of the external treatment. The plastically-compacted collagen gel may also be said to be dehydrated.

In contrast to prior art collagen gels such as those produced under the trade mark Gelfoam® (which are said to be capable of absorbing 45 times their weight in blood), the plastically-compacted collagen gels of the invention are permanently compressed and are essentially non-absorbable. In this context, the term “plastically compacted” means that the compaction results in a permanent compression/distortion of the structure of the gel.

The plastically-compacted gels referred to herein are not vitrified (i.e. they are not dried to an extent which produces a rigid, glass-like material); they are not glass-like; they are not rigid; they are flexible. The collagen gels used here are capable of, having live cells such as fibroblasts and/or keratocytes entrapped within their structure.

The collagen which is used in the collagen gel may be any fibril-forming collagen. Examples of fibril-forming collagens are Types I, II, III, V, VI, IX and XI. The gel may comprise all one type of collagen or a mixture of different types of collagen. Preferably, the gel comprises or consists of Type I collagen. In some embodiments of the invention, the gel is formed exclusively or substantially from collagen fibrils, i.e. collagen fibrils are the only or substantially the only polymers in the gel.

In other embodiments of the invention, the collagen gel may additionally comprise other naturally-occurring polymers, e.g. silk, fibronectin, elastin, chitin and/or cellulose. Generally, the amounts of the non-collagen naturally-occurring polymers will be less than 5%, preferably less than 4%, 3%, 2% or 1% of the gel (wt/wt). Similar amounts of non-natural polymers may also be present in the gel, e.g. polylactone, polylactide, polyglycone, polycapryolactone and/or phosphate glass.

The interstitial liquid may be any liquid in which collagen fibrils may be dissolved and in which the collagen, fibrils may gel. Generally, it will be an aqueous liquid, for example an aqueous buffer or cell culture medium.

In some embodiments of the invention, one or more surfaces of the collagen gel are coated with laminin, or one or more laminin domains, in order to improve the adherence of corneal cells. Laminin, an extracellular matrix (ECM) multidomain trimeric glycoprotein, is the major non-collagenous component of basal lamina that supports adhesion, proliferation and differentiation. It was initially isolated from mouse Engelbreth-Holm-Swarm (EHS) tumor (laminin-1). Laminin proteins are integral components of structural scaffolding in animal tissues. Laminins associate with type IV collagen via entactin and perlecan and bind to cell membranes through integrin receptors, dystrogylcan glycoprotein complex and Lutheran blood group glycoprotein.

As used herein, the term “laminin domain” includes, inter alia, RGD and IKVAV sequences of the α-chain, YIGSR of the β1-chain, and RNIAEIIKDI of the γ-chain.

Preferably, the laminin is from Engelbreth-Holm-Swarm murine sarcoma basement membrane.

The laminin or laminin domains may, for example, be used at a concentration of 1-2 μg/cm². The laminin or laminin domains be may applied to the collagen gel before or after compaction. Preferably, only the surface onto which the corneal cells are placed is coated. This may, for example, be the upper surface (when in use) of the collagen gel.

In some embodiments, the uncompacted collagen gel may comprise no cells within the gel. In yet other embodiments, the uncompacted collagen gel may comprise one or more types of cells. Examples of such seeded cells include stromal progenitor cells such as corneal fibroblasts (keratocytes) in an differentiated or undifferentiated form. Preferably, these corneal fibroblasts are obtained from the peripheral limbus or from limbal rings which are incubated overnight with about 0.02% collagenase at about 37° C.

Such cells, if present, are generally seeded into the collagen gel prior to compaction (i.e. dehydration), for example, by mixing them with the collagen solution prior to polymerization/aggregation.

Examples of suitable methods of gel compaction (with or without cells in the gel) include the following:
(i) the application of a compressing force to one or more of the surfaces or edges of the gel;
(ii) the application of a dehydrating force to one or more of the surfaces or edges of the gel;
(iii) the stretching of the gel in one or two planes (e.g. length and/or width); or
(iv) a combination of one or more of (i)-(iii).

Each of the aforementioned methods may be combined with the direct application (i.e. contact) of an interstitial liquid-absorbing material to one or more of the surfaces or edges of the gel.

In some embodiments, the compaction of the collagen gel may have been produced by applying a compressing force to one or more surfaces or edges of the gel. Preferably, the gel is confined during the application of the compressing force. Preferably, the compressing force is applied to the upper surface of the gel. For example, a weight may be applied to the upper surface of the gel, optionally together with the application of an interstitial liquid-absorbing material to the gel. The amount of the weight and the duration of compression will vary depending on the level of the desired compaction. In some embodiments, the weight will be 20-100 g, preferably 40-60 g, most preferably about 50 g. In some embodiments, the duration of compression will be 10-600 seconds, preferably 20-400 seconds, most preferably about 5 minutes.

In other embodiments, the compaction of the collagen gel may have been produced by applying a dehydrating force to one or more surfaces or edges of the gel. For example, interstitial liquid-absorbing material may be applied to the upper and/or lower surfaces of the gel. Examples of such liquid-absorbing-materials include one or more sheets of tissues and blotting paper. The duration of the application of the interstitial liquid-absorbing material will vary depending on the level of the desired compaction.

In yet other embodiments, the compaction of the collagen gel may have been produced by stretching of the gel in one or two planes (e.g. length and/or width). The effect of such stretching may be to force out a portion of the interstitial liquid. For example, the gel may be suspended from a first edge and a load is applied to a second (e.g. opposite) edge. The load will be of an amount which is capable of stretching the gel without breaking the gel. Different loads may be applied across different axes of the gel. The duration of the application of the load(s) and the amount of the load(s) will vary depending on the level of the desired compaction. In a preferred embodiment, an interstitial liquid-withdrawing force or dehydrating force may be applied along the same axis as the load, for example by an interstitial liquid-absorbing material being placed at one or both edges of the gel to which loads are applied.

Before or after the compaction of the gel, the gel may be subjected one or more repetitive cycles of (a) applying a uniaxial tensile load and (b) removing the said load. It is believed that such repetitive cycles of loading and unloading increases fusion of collagen fibrils in the compacted gel in an oriented manner (see, for example, WO2007/060459).

Further methods for the production of compacted collagen gels are known in the art (e.g. WO2006/003442, WO2007/060459 and WO2009/004351).

Under the external compacting/dehydrating treatment, interstitial liquid is permanently removed from the compacted gel. The resultant gel has a permanently-reduced volume, increased density and increased strength compared to the original (uncompacted) gel.

The volume of the collagen gel might, for example, have been reduced by at least 50%, 60%, 70%, 80%, 90%, 95%, 99% or 99.9%. Preferably, the volume of the gel is 0.1-2.0% of the original volume.

The time required to effect compaction may vary depending on the applied external treatment. For example, compaction may be effected in less than 24 hours, less than 12 hours, less than 6 hours, less than 3 hours or less than 1 hour. In other embodiments, compaction may be effected in less than 30, 20, 10, 5 or 2 minutes.

The amount of interstitial liquid lost from the gel, compared to that in the original gel, may be at least 50%, 60%, 70%, 80%, 90%, 95%, 96%, 97%, 98% or 99%.

For the production of artificial ocular epithelia for grafting or for oculotoxicity testing or any other uses disclosed herein, the plastically-compacted collagen gel will preferably be 1-60 mm long, and more preferably 20-40 mm long. It may also be 0.5-60 mm wide, and preferably 20-40 mm wide.

In some embodiments of the invention, the plastically-compacted collagen gel will be in the form of a sheet which is 5-10000 μm thick, preferably 10-1000 μm, more preferably 20-100 μm thick, and most preferably about 50 μm thick.

The composition of the plastically-compacted collagen gel is generally 3-4% collagen (preferably 3.3-3.5%, more preferably about 3.4% collagen), with the remainder being water and salts/sugars from the buffer. Of this remainder, water will typically constitute >99%.

The diameter of the collagen fibrils in the compacted collagen gels is preferably 10-100 nm. The spacing of the collagen fibrils in the compacted collagen gels are preferably 1-200 nm. These parameters may be measured by the following method: Collagen gels may be fixed in 2.5% glutaraldehyde in PBS for 1 hour at room temperature followed by 1% osmium tetroxide for 1 hour at room temperature, then dehydrated in increasing ethanol concentrations (up to 100%) followed by gassing in propylene oxide then embedding in Agar 100 resin polymerised at 60° C. for 24 hours. 70 nm sections may be cut and counter-stained by lead citrate and uranyl acetate before examination in a transmission electron microscope (TEM), where collagen fibril diameter and spacing may be quantified. The orientation of collagen fibrils may also be assessed qualitatively, e.g. high (or low) degree of orientation, by this method.

In another aspect, the invention provides the use of a plastically-compacted collagen gel as a substrate upon which to grow corneal cells.

The invention also provides a process for producing an artificial ocular epithelium comprising culturing corneal stem cells or a composition comprising corneal stem cells on a plastically-compacted collagen gel substrate, wherein the cells or the composition are cultured under conditions such as to provide a population of corneal epithelial cells which produce an artificial ocular epithelium on the substrate.

The plastically compacted gel used in the invention provides a substrate for the corneal cells to grow upon, this substrate being similar in morphology to denuded corneal stroma. The cells grow on the surface of this substrate, with no or essentially no growth of such cells into the substrate. The level of compaction of the plastically-compacted collagen gel is such that it prevents ingrowth of the applied epithelial cells into the compacted gel.

In some embodiments, the artificial ocular epithelium is subsequently isolated from the substrate.

In other embodiments of the invention, the artificial ocular epithelium is retained on the plastically-compacted collagen gel substrate and the latter is used as an artificial corneal stroma. As used herein, the term “artificial corneal stroma” refers preferably to a plastically compacted collagen gel as herein defined, which may optionally comprise corneal fibroblasts and/or ketatinocytes entrapped therein, and/or which may optionally be cross-linked (preferably using riboflavin/UV).

In some embodiments, the artificial ocular epithelium is subsequently stored in media suitable for the storage and preservation of human tissue, with or without the substrate, preferably a chondroitin-sulphate-based storage media, e.g. Optisol® (Bausch & Lomb), optionally together with instructions for use as an artificial ocular epithelium.

Preferably, the plastically-compacted collagen gel substrate is obtained or obtainable by a process as described herein.

The invention also provides an artificial ocular epithelium obtained or obtainable by the above process.

The invention also provides an artificial ocular epithelium comprising a continuous stratified epithelium of 3-7 cell layers expressing both CK3 (cytokeratin 3) differentiation marker and CK14 (cytokeratin 14) undifferentiation marker with basal membrane components (e.g. laminin, integrins, hemidesmosomes) within and beneath the basal cells, preferably obtained by or obtainable by a process as defined herein.

The artificial ocular epithelium preferably has an optical density (OD) of 0.00-0.50 at 450 nm. Preferably the laminin-coated plastically-compacted collagen gel with embedded keratinocytes and artificial ocular epithelium has an OD (450 nm) of 0.01-0.10, preferably about 0.073.

The presence of desmosomes and hemidesomosomes (cell-cell and cell-substrate adhesion complexes, respectively) in the artificial ocular epithelium can be used to quantify tissue integrity and adhesion to the underlying matrix. In particular, the invention relates to artificial ocular epithelia wherein hemidesmosomes are present in some or all basal cells and/or some or all neighbouring epithelial cells are attached to each other via desmosome structures.

The composition comprising corneal stem cells preferably compries limbal epithelial cells, i.e. a heterogeneous mixture of stem cells and differentiated cells which is obtainable from the limbus at the edge of the cornea. In other words, the composition comprising corneal stem cells may comprise a mixture of corneal stem cells and cells that have not yet fully committed to a corneal epithelial phenotype.

As used herein, the term “corneal cells” refers to cells which have been obtained from an animal (preferably mammalian) cornea. Preferably, the cells are obtained from the limbal ring of the cornea, i.e. the outer edge of the cornea excluding the conjunctiva, iris and central cornea. The cells may comprise or consist of epithelial cells. The cells may comprise or consist of corneal stem cells, preferably limbal corneal epithelial stem cells. Preferably, the corneal stem cells are human corneal stem cells.

The collagen in the compacted collagen gels may be cross-linked before or after compaction in order to improve the mechanical properties of the gels. Preferably, the cross-linking is performed using riboflavin and UV (preferably UVA, most preferably at about 365nm). For example, the cross-linking may be performed by incubating the compacted gel in a riboflavin solution (preferably 0.05-0.2% riboflavin in a 15-25% dextran solution) for 20-40 minutes at room temperature. Any unused riboflavin may then be washed out of the gel, e.g. using PBS. Collagen gels treated in this way are capable of withstanding an increased load compared to non-treated gels and are better held in place by sutures when transplanted to the ocular surface.

In some embodiments of the invention, the cross-linking is not performed using 1-ethyl-3-(3-dimethyl aminopropyl) carbodiimide (EDC) or N-hydroxysuccinimide (NHS) or any other carbodiimide- or succinimide-based cross-linking agents.

In one preferred embodiment of the invention, a cross-linked plastically compacted collagen gel is used (preferably cross-linked using riboflavin/UV), wherein the compacted gel does not comprise entrapped cells.

The invention also provides a plastically-compacted collagen gel, wherein the collagen fibres have been cross-linked using riboflavin (preferably using UV light), and uses of such gels as a substrate upon which an artificial ocular epithelium may be grown, and for the other uses disclosed for herein. Preferably, the plastically-compacted collagen gel is one produced by a process as disclosed herein.

The composition or stem cells are cultured under conditions such as to provide a population of corneal epithelial cells which produce an artificial ocular epithelium on the surface of the substrate. Such conditions are well known in the art (e.g. Ebato B., et al. Invest. Opthalmol. Vis. Sci. 1988; 29:1533-1537; de Paiva C. S. et al. Stem Cells 2005; 23:63-73).

The invention further provides an artificial ocular tissue comprising an artificial ocular epithelium of the invention and a plastically-compacted collagen gel substrate obtained by or obtainable by a process of the invention, preferably wherein the artificial ocular epithelium is growing or has grown on the surface of the plastically-compacted collagen gel substrate.

The invention further provides a method of assessing the effect of a test compound on an artificial ocular epithelium or artificial ocular tissue, comprising the steps:
(a) providing an artificial ocular epithelium or tissue obtained by or obtainable by a process of the invention;
(b) contacting the artificial ocular epithelium or tissue with an amount of the test compound; and
(c) assessing the effect of the compound on the artificial ocular epithelium or tissue.

The effect of the compound may, for example, be assessed by any analytical, biochemical, optical, microscopic or other means.

In some embodiments, the effect to be assessed is a change in optical character of the artificial ocular epithelium or tissue, or a change in the permeability of the artificial ocular epithelium or tissue. The change may, for example, be measured before and after the application of the test compound or the change may be compared to a control.

In other embodiments, the effect of the compound may be assessed by histological examination of the artificial ocular epithelium or tissue, or by measuring the production of any pro-inflammatory mediator.

The invention also provides the use of an artificial ocular epithelium or tissue obtained by or obtainable by a process of the invention for providing an indication of the toxicity of the test compound on the mammalian cornea.

In other embodiments, the invention provides the use of an ocular epithelium or tissue obtained by or obtainable by a process of the invention for providing a model to investigate underlying/basic biology of corneal epithelium, e.g. molecular control of proliferation, differentiation, attachment and stratification.

The invention also provides the use of an artificial ocular epithelium or tissue obtained by or obtainable by a process of the invention as an artificial cornea.

The invention also provides the use of an artificial ocular epithelium or tissue obtained by or obtainable by a process of the invention as an agent for the delivery of cells to a tissue in need thereof.

The invention further provides a method of treating an ocular injury comprising:
(a) providing an artificial ocular epithelium or tissue obtained by or obtainable by a process of the invention;
(b) contacting the ocular injury with said artificial ocular epithelium or tissue; and optionally
(c) securing the said artificial ocular epithelium or tissue at the site of the ocular injury.

Ocular injuries that might be treated include those related to an insufficient stromal micro-environment to support stem cell function, such as aniridia, keratitis, neurotrophic keratopathy and chronic limbitis; or related to external factors that destroy limbal stem cells such as chemical or thermal injuries, Stevens-Johnson syndrome, ocular cicatricial pemphigoid, contact lens wear, or extensive microbial infection.

The invention further provides an artificial ocular epithelium or tissue obtained by or obtainable by the above process for use in a method of therapy, preferably in a method of treating ocular injuries such as those defined above.

The invention also provides the use of an artificial ocular epithelium or tissue obtained by or obtainable by the above process in the manufacture of a composition for a method of therapy, preferably in a method of treating ocular injuries such as those defined above.

The invention also provides a method of replacing a cornea in an mammalian subject comprising:
(a) providing an artificial ocular epithelium or tissue obtained by or obtainable by a process of the invention;
(b) replacing the cornea of the mammalian subject with said artificial ocular epithelium or tissue.

The invention also provides an artificial ocular epithelium or tissue obtained by or obtainable by the above process for use in a method of surgery, preferably wherein the cornea of a mammalian subject is replaced with said artificial ocular epithelium or tissue.

The invention also provides the use of an artificial ocular epithelium or tissue obtained by or obtainable by the above process in the manufacture of a composition for a method of surgery, preferably wherein the cornea of a mammalian subject is replaced with said artificial ocular epithelium or tissue.

### BRIEF DESCRIPTION OF THE FIGURES

FIG. 1. Primary sphere formation by keratocytes from the limbus of the bovine corneal stroma. The representative spheres cultured 5 (A), 7 (B) and 9 (C) days respectively. (D): The differentiated progeny from the primary sphere. The scale bar=50 μm.

FIG. 2. Live/dead staining of the embedded keratocytes. A: Embedded keratocytes within compressed collagen gel after 7 days in culture. B: The keratocytes were alive indicated by their green staining. C: Dead keratocytes showing red staining were not detected.

FIGS. 3A-B. Transmission electron microscopy (TEM) of human cornea (FIG. 3A) and amniotic membrane (FIG. 3B).

FIGS. 4A-B. X-ray diffraction of amniotic membrane (FIG. 4A) showing the transect across the X-ray diffraction pattern (FIG. 4B).

FIG. 5. Scanning electron micrographs of different scaffolds. A: compressed collagen gel; B: denuded amniotic membrane.

FIG. 6. Transmission electron microscope images of corneal epithelia sheets and normal bovine corneal epithelium. A: Basal cells appeared to adhere well to the compressed collagen scaffold via hemidesmosome attachments (arrows); B: Hemidesmosome attachments in normal bovine corneal epithelium (arrows); C: Neighbouring cells clearly displayed desmosome junctions (arrows) on compressed gels; D: Desmosome junctions in normal corneal epithelium (arrows). Scale bars: 800 nm.

FIG. 7. Evaluation of transparency. A: Line 1 (“colllagen”) laminin coated compressed collagen gel with embedded keratocytes; line 2 (“AM”) denuded amniotic membrane; line 3 (“collagen+”) combination of LECs expanded upon compressed collagen gel; line 4 (“AM+”) combination of LECs expanded upon denuded amniotic membrane. Tissue placed in a 96 well plate. B: The resulting OD measurements. Bar chart represents the mean and standard deviation.

FIGS. 8A-C. Stratification of isolated limbal cells on amniotic membrane. Expanded cells from limbal pieces after 11 days in basal culture media incubation (A) and suspended cells after 14 days (B). Expanded cells on dehydrated collagen sheet showing comparable level of cell density and stratification (C). Staining indicates cell nuclei.

FIGS. 9A-B. 20× photomicrograph of K3 (Red) and K14 (Green), DAPI (blue) double labelling of corneal limbal cells after 11 days culturing. Suspension cultured cells (A) and Explant cultured cells (B) Scale bar: 100 μm

FIG. 10. Immunofluorescent staining of expanded limbal epithelial cells. A: CK3 staining (green) of LECs (red) on laminin coated compressed collagen gel embedded with keratocytes. B: CK3 staining (red) of LECs (blue) on denuded Amniotic membrane. C: CK14 (green) staining of LECs (red) on laminin coated compressed collagen gel embedded with keratocytes. D: CK14 (green) staining of LECs (blue) on denuded amniotic membrane. Scale bar=50 μm.

FIG. 11. Western blotting and immunoblotting of CK3 (A—“K3”) and CK14 (B—“K4”) expression of LECs cultured on laminin coated compressed collagen gel embedded with keratocytes (Collagen) and denuded amniotic membrane (AM).

FIG. 12. CK12 mRNA expression in LECs cultured on laminin coated compressed collagen gel embedded with keratocytes (collagen) and denuded amniotic membrane (AM).

FIG. 13. Plastic compression of collagen gels. A: A stabilized uncompressed collagen gel; B: Diagram showing the method for PC of stabilized collagen gels; C: A compressed collagen gel.

FIG. 14. Limbal epithelial outgrowths. A: Explant outgrowths on uncompressed collagen gel; B: Explant outgrowths on compressed collagen gel; C: Graph showing the area of explant outgrowth on collagen scaffolds. Scar bar=50 μm.

FIG. 15. Scanning electron micrographs of different scaffolds. A: Uncompressed collagen gel; B: Compressed collagen gel; C: Denuded bovine corneal stroma.

FIG. 16. Scanning electron microscope of LECs on collagen gel and normal cornea. A: Cells on uncompressed collagen gel; B: Cells on compressed collagen gel; C: Normal bovine corneal epithelium.

FIG. 17. Transmission electron microscope images of collagen fibres, corneal epithelia sheets and normal bovine corneal epithelium. A-C collagen fibres from different scaffolds: uncompressed collagen gel (A), compressed collagen gel (B) and normal bovine corneal stroma (C); D: Basal cells do not adhere very well to the uncompressed collagen gel; E: Basal cells appear to adhere well to the compressed collagen scaffold via hemidesmosome attachments (arrows); F: Hemidesmosome attachments in normal bovine corneal epithelium (arrows); G: Large gaps between cell layers are visible on uncompressed gels (arrows); H: Neighbouring cells clearly display desmosome junctions (arrows) on compressed gels; I: Desmosome junctions in normal corneal epithelium (arrows). Scale bars: (A-C) 10 μm; (D-I) 1 μm.

FIG. 18. Immunostaining of cells grown on collagen gels and normal bovine corneal epithelium. Propidium iodide (red) and CK 3 (green). A: Cells grew on uncompressed collagen gel; B: Cells grew on compressed collagen gel; C: Normal bovine cornea epithelium. Scale bar=50 μm.

FIG. 19. Compressed collagen gels which are untreated (left) and riboflavin/UV treated (right).

FIG. 20. Equipment used to analyse the breaking strain of compressed collagen gels.

FIG. 21. Examples of increasing load against time for untreated (FIG. 21A) and riboflavin/UV treated (FIG. 21B) compressed collagen gels.

FIG. 22. Immunostaining of cells grown on riboflavin/UV treated collagen gels. Propidium iodide (red) and CK 3 (green). Corneal limbal cells can grow across the riboflavin treated compressed collagen gel.

FIG. 23 Compressed collagen gels transplanted on to the ocular surface of a rabbit (lamellar graft). A: a compressed collagen gel once transplanted does not hold sutures efficiently; B: a compressed collagen gel with riboflavin treatment enables improved transplantation as it can be better sutured and held in place.

### EXAMPLES

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims. The disclosure of each reference set forth herein is incorporated herein by reference in its entirety.

### Example 1

### Isolation of Keratocytes using a Primary Sphere Forming Assay

Normal bovine eyes were obtained from a local abattoir (Chity wholesale abattoir, Guildford, UK) within 2 hours of death, transported to the laboratory at 4° C. and used immediately. Corneoscleral buttons were dissected using standard eye bank techniques. Briefly, corneoscleral tissues were rinsed three times with Dulbecco's minimal essential medium (DMEM, GIBCO). After careful removal of the central cornea, excess sclera, iris, corneal endothelium, conjunctiva and Tenon's capsule the remaining limbal rims were cut into small pieces approximately 25 mm². From these pieces the limbal stromal keratocytes and epithelial cells were subsequently isolated. For limbal stromal keratocyte isolation the pieces of limbal rims were incubated with 0.02% collagenase (GIBCO) at 37° C. overnight. The remaining limbal stromal pieces were then collected and treated with 0.2% EDTA (Sigma, UK) at 37° C. for 5 min then aspirated through a 21 guage needle to isolate into single cells. After centrifugation, the cells were resuspended in basal medium containing DMEM and Ham's F12 medium (DMEM/F12,1:1) supplemented with B27 (Invitrogen, UK), 20 ng/ml epidermal growth factor (EGF, Sigma, UK), 40 ng/ml basic fibroblast growth factor Mary Ann Liebert, Inc.,140 Huguenot Street, New Rochelle, N.Y. 10801 (bFGF, Sigma, UK), 100 U/ml penicillin, 100 μg/ml streptomycin, and 250 ng/ml amphotericin B. A sphere-forming assay was employed to culture these isolated limbal keratocytes using basal medium containing methylcellulose gel matrix (0.8%, Sigma-Aldrich). Plating was done at a density of ten viable cells/μl in 60 mm culture dishes 27.

### Example 2

### Differentiation of Sphere Colonies

Primary spheres formed from the suspended limbal keratocytes and after 7 days in culture were transferred to glass coverslips coated with 50 m/ml poly-L-lysine (Sigma, UK) and 10 μg/ml fibronectin (Sigma, UK) for microscopic investigation. To promote differentiation of the limbal keratocytes, 1% fetal bovine serum was added to the basal medium, and the culture was continued for seven days. The resulting differentiated keratocytes were digested in 0.25% trypsin and 0.02% EDTA (Sigma, UK) and resuspended in basal media at a density of 2.0×10⁵ cells.

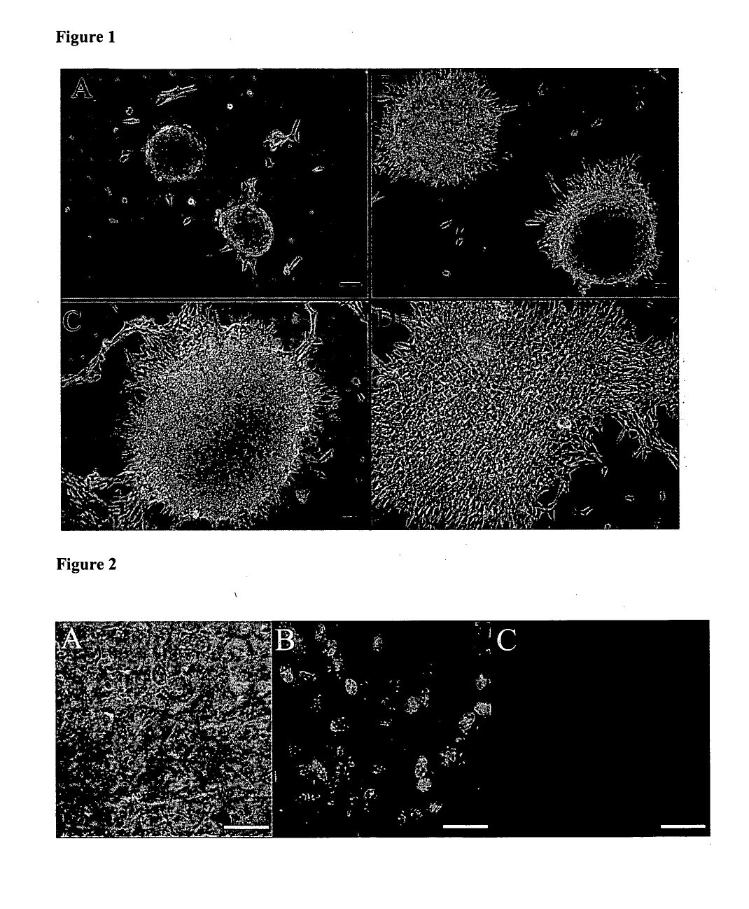

When the limbal stroma was disaggregated into single cells and cultured for nine days, viable spheres of cells grew during this period. Photographs of representative spheres cultured 5, 7, 9 days are shown in FIGS. 1A, 1B and 1C, respectively. The differentiated progeny from each primary sphere showed a typical fibroblast-like morphology (FIG. 1D).

### Example 3

### Formation of Acellular Collagen Gels

Acellular collagen gels were made, as described previously (Brown et al., Adv. Funct. Mater., 2005, 15: 1762-1770) by neutralizing 4 mL of sterile rat-tail type I collagen (First Link Ltd. West Midlands, UK) in 1 mL of 10× concentration Eagle minimum essential medium (Gibco, Paisley, UK) with 0.5 mL 1 Mol sodium hydroxide (Merck, Leicestershire, UK). Gels were cast into rectangular moulds (33 mm×13 mm×4 mm) and set/stabilized in a 37° C. 0.5% CO₂ incubator for 30 min. Following setting and incubation, gels were compacted by a combination of compression and blotting using layer of nylon mesh and paper sheets (an additional metal wire mesh used by Brown et al. was not used). To achieve compaction of the gels, a layer of nylon mesh (50 μm mesh size) was placed on a double layer of absorbent paper, the collagen gel was placed on the nylon mesh and covered with a second nylon mesh, and loaded with a 50 g weight for 5 min at room temperature, leading to the formation of a flat collagen sheet (20-40 μm thick) protected between two nylon meshes.

### Example 4

### Formation of Collagen Gels Loaded with Fibroblasts

A pellet of stromal fibroblasts extracted from fresh corneal tissue or fibroblastic cell line is suspended in 4 mL of sterile rat-tail type I collagen (First Link Ltd. West Midlands, UK) in 1 mL of 10 × concentration Eagle minimum essential medium (Gibco, Paisley, UK), and is neutralised with 0.5 mL 1 Mol sodium hydroxide (Merck, Leicestershire, UK). The gels containing cells are cast into rectangular moulds (33 mm×13 mm×4 mm) and set/stabilized in a 37° C. 0.5% CO₂ incubator for 30 min. Following setting and incubation, gels are compacted by a combination of compression and blotting using layer of nylon mesh and sheets of filter paper. To achieve compaction of the gels a layer of nylon mesh (50 μm mesh size) is placed on a double layer of absorbent paper, the collagen gel is placed on the nylon mesh and covered with a second nylon mesh, and loaded with a 50 g weight for 5 min at room temperature, leading to the formation of a flat collagen sheet (20-40 μm thick) protected between two nylon meshes.

### Example 5

### Laminin Coating of Collagen Gels

In some cases, the resulting compressed collagen gels, embedded with keratocytes, were then transferred into 6 well plates (transwells, costar) and each gel coated with laminin solution (50 μg/ml, Sigma, UK), incubated at 37° C. for 2 hours, washed 3 times with phosphate buffered saline (PBS) at which point the collagen scaffolds were ready for LECs expansion.

### Example 6

### Assay for Keratocyte Survival

The survival of the keratocytes embedded within the compressed gel was examined using a live/dead double staining kit (Calbiochem, German) following 7 days cultured in DMEM and Ham's F12 (DMEM/F12) medium, supplemented with 10% FBS (Sigma, UK), 0.5% DMSO (Sigma,UK), 10 ng/ml EGF (Sigma,UK), 5 mg/ml insulin (Sigma,UK), 100 IU/ml penicillin and 100 mg/ml streptomycin. The kit utilizes cyto-dye, a cell-permeable green fluorescent dye to stain live cells whilst the dead cells were stained by propidium iodide (PI), a non-permeable red fluorescent dye that can only enter the cell when there is membrane damage that results in permeabilization. A confocal microscope (LEICA DMIRE2, German) was used to detect the ratio of live to dead keratocytes.

The embedded keratocytes were cultured for 7 days, during which time the collagen gel did not noticeably change its dimensions. The cells within the gel were treated to live/dead double staining and examined by confocal microscopy (FIG. 2A). By focusing at various depths through the gel we detected that the cells remained viable (FIG. 2B) and no dead cells were seen (FIG. 2C), indicating that the encapsulation and subsequent compression of keratocytes within the collagen gel did not affect cell viability during this period.

### Example 7

### Structural Details of Cornea, Amniotic Membrane and Collagen Gels

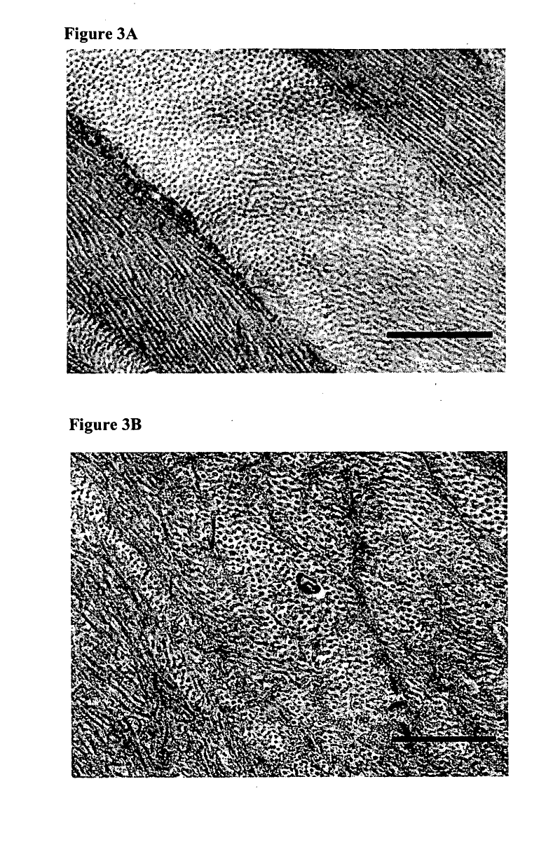

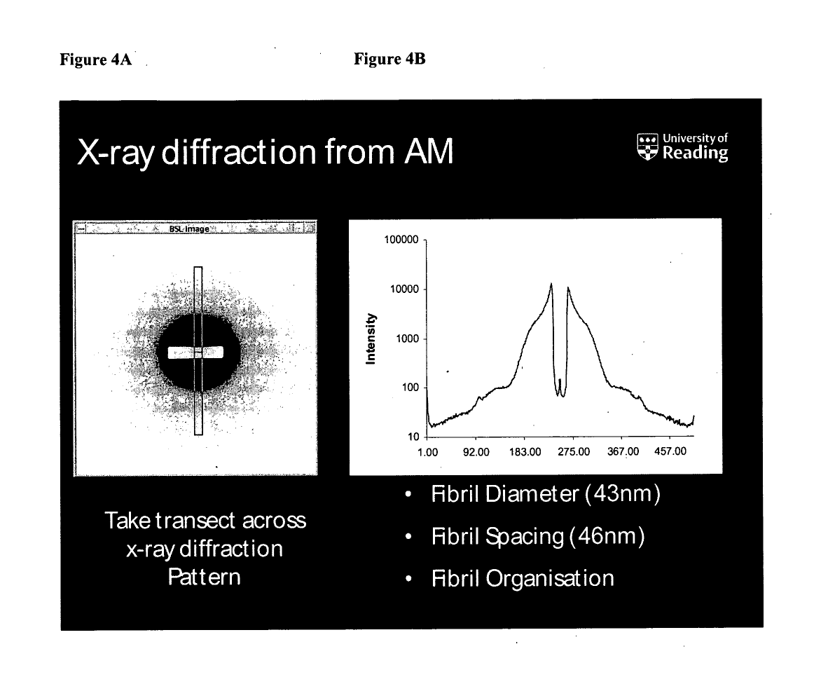

Transmission electron microscopy (TEM) of human cornea and amniotic membrane revealed similarity of structure in terms of collagen fibre diameter, spacing and orientation. (FIGS. 3, A and B). X-ray diffraction of amniotic membrane revealed a fibril diameter of 43 nm, a fibril spacing of 46 nm and illustrated the fibril organisation (FIG. 4).

### Example 8

### Preparation of Cell Suspension from Limbal Cells

Limbal ring at the outer edge of the cornea was dissected from the conjunctiva, iris and central cornea, maintaining the limbal ring structure for limbal epithelial cell isolation. The limbal ring was cut into several pieces, approximately 1 cm long, which were incubated for 12 hours at 37° C. with 0.02% type IA collagenase (Sigma-Aldrich) in basal culture medium containing DMEM, FM12 (1:1) media (Fisher Sci, U.K.), 50 μg/ml antibiotics, 5% FBS, 0.5% dimethyl sulfoxide, 2 ng/ml human Epidermal Growth Factor, 5 μg/ml insulin, B27 supplement medium (Fisher Sci, U.K.), in an atmosphere of humidified 5% carbon dioxide and 95% air, at 37° C.

Epithelial sheets were peeled off from the enzyme-incubated limbal pieces by fine forceps, then were transferred into 15 ml tubes containing 0.05% trypsin/EDTA for 10 to 15 minutes incubation at 37° C., and finally dissociated into single cells by agitation through a 21 gauge needle. Trypsin/EDTA was removed by adding basal culture medium with FBS and followed by several rounds of centrifugation 1000 rpm for 5 mins at room temperature. Cells were resuspended in basal culture medium and seeded onto a collagen gel or amniotic membrane.

### Example 9

### Preparation of Explant Containing Limbal Stem Cells

The limbal ring structure was cut equally into 8-10 pieces, each of these measuring 5 mm×5 mm square, finally the underlying limbal stroma (approximately two thirds of the thickness of stroma) was also carefully removed. The limbal pieces were washed 3 times with sterile PBS and followed by rinsing in a penicillin/streptomycin antibiotics solution (Gibco) for 3mins. The limbal corneal limbal pieces were placed on to a Petri dish epithelial side up, submerged with basal culture medium. The limbal pieces were incubated in an atmosphere of humidified 5% carbon dioxide and 95% air, at 37° C. for 2-3 days. Once the limbal epithelial cells could be seen to be migrating down the edge of the limbal explants (by inverted light microscope) on to the Petri dishes they were deemed ‘healthy’ and suitable for further cultivation. Such limbal pieces, were carefully removed from the plastic dish and gently transferred to a substrate (collagen gel or amniotic membrane) by culture inserts within a covered 6 well plate.

### Example 10

### Expansion of Limbal Epithelial Cells on Compressed Collagen Gels and Denuded Amniotic Membrane

The amniotic membrane (AM) was washed three times with sterilized PBS buffer, then treated with 0.25% trypsin at 37° C. for 30 min. After the incubation, the epithelial cells on the membrane were removed with a scraper. The cell-free AM was then transferred into 6 transwells with the basement membrane surface upwards. The isolated LSCs were seeded onto laminin coated compressed collagen gel with embedded keratocytes and denuded AM at 10⁶ cells/ml. After 14 days the expanded LECs were exposed to air by lowering the medium level for a further 7 days. After 3 weeks incubation the corneal epithelium membrane with multiple layers of cells was ready for further examination.

### Example 11

### Electron Microscopy

The surfaces of compressed collagen gel and denuded AM were examined by scanning electron microscopy (SEM). LEC's expanded upon compressed collagen gels were examined by transmission electron microscopy (TEM). All specimens were fixed in 2.5% (v/v) glutaraldehyde, washed three times for 10 minutes in PBS, and post-fixed for 2 hours in 1% aqueous osmium tetroxide. Specimens were then washed 3 more times in PBS before being passed through a graded ethanol series (50%, 70%, 90% and 100%). For SEM, specimens were transferred to hexamethyldisilazane for 20 minutes and allowed to air dry. These specimens were then mounted on aluminium stubs and sputter coated with gold before examination using an SEM (FEI Quanta FEG 600, UK). For TEM, the dehydrated specimens were embedded in epoxy resin (Agar 100; Agar Scientific, Ltd., Stansted, UK). Ultrathin (70 nm) sections were collected on copper grids and stained for 1 hr with uranyl acetate and 1% phosphotungstic acid and then for 20 min with Reynolds' lead citrate before examination using a transmission electron microscope (Philips CM20, Holland).

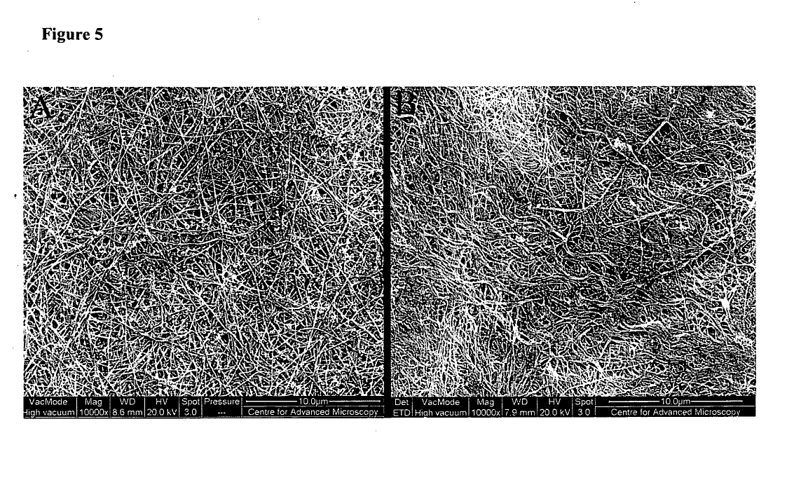

The SEM analyses of the collagen fibres within the compressed gel (FIG. 5A) appeared dense and homogeneous, similar in morphology and structure to the denuded AM (FIG. 5B).

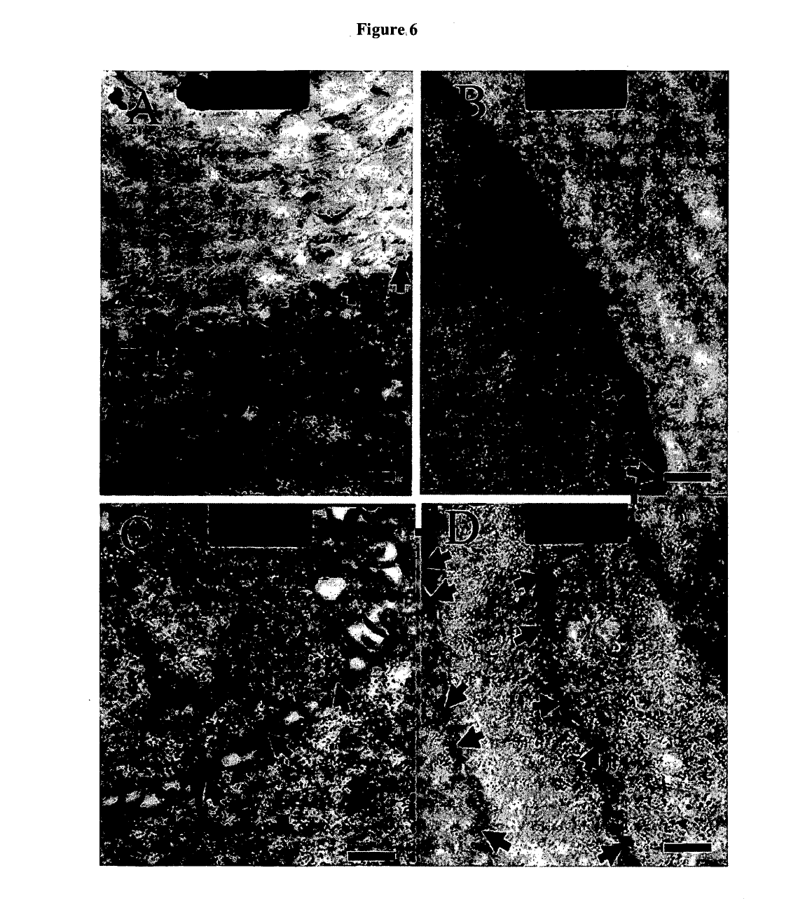

TEM analyses indicated that the LECs once expanded upon a compressed gel produced a defined basement membrane layer with evidence of hemidesmosome formation in the basal cells (FIG. 6A), similar to that shown by normal corneal epithelium (FIG. 6B). Furthermore, neighbouring cells were attached via desmosome structures (FIG. 6C), again similar to that seen in normal corneal epithelium (FIG. 6D).

### Example 12

### Assessment of Transparency

To assess the transparency of both compressed collagen gel and denuded AM, before and after LEC's expansion, the resultant corneal constructs were dissected into 3.5-mm diameter pieces using a trephine and placed individually into the wells of 96-well culture plates. A Bio-Tek Instrument (E1x800UV, UK) was used to measure the tissues optical density (OD).

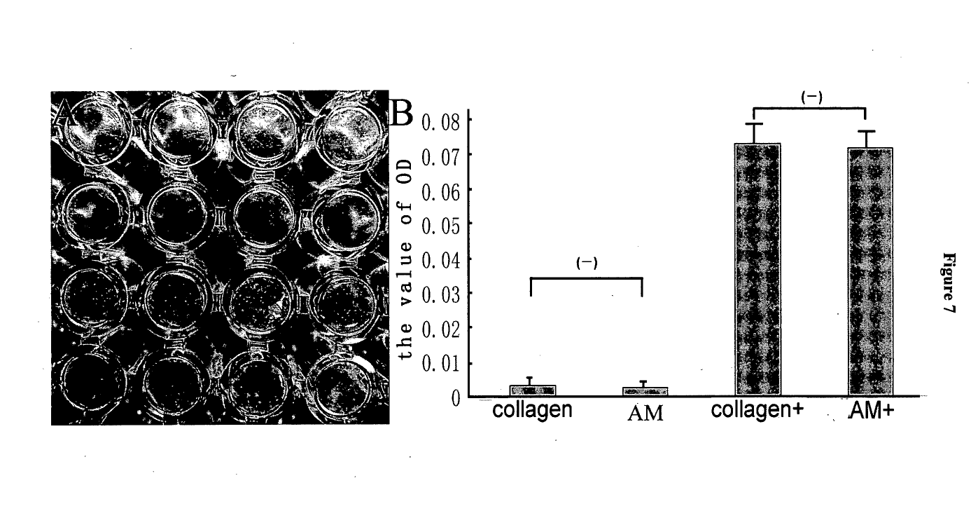

Optical density (OD at 450 nm) measurements were taken to facilitate a comparison in transparency between LECs grown on compressed collagen gel and denuded AM (FIG. 7A). The OD values from laminin coated compressed collagen gel embedded with keratocytes (0.003±0.001;) and denuded AM (0.003±0.001) were very low, and there were no significant differences between them (P>0.05). The OD values taken from the laminin coated compressed collagen gel embedded with keratocytes and denuded AM, each following the addition of expanded LECs, were 0.073±0.003 and 0.072±0.003 respectively, with no significant difference between them (P>0.05) (FIG. 7B).

### Example 13

### Stratification of Limbal Cells on Collagen Gel or Amniotic Membrane

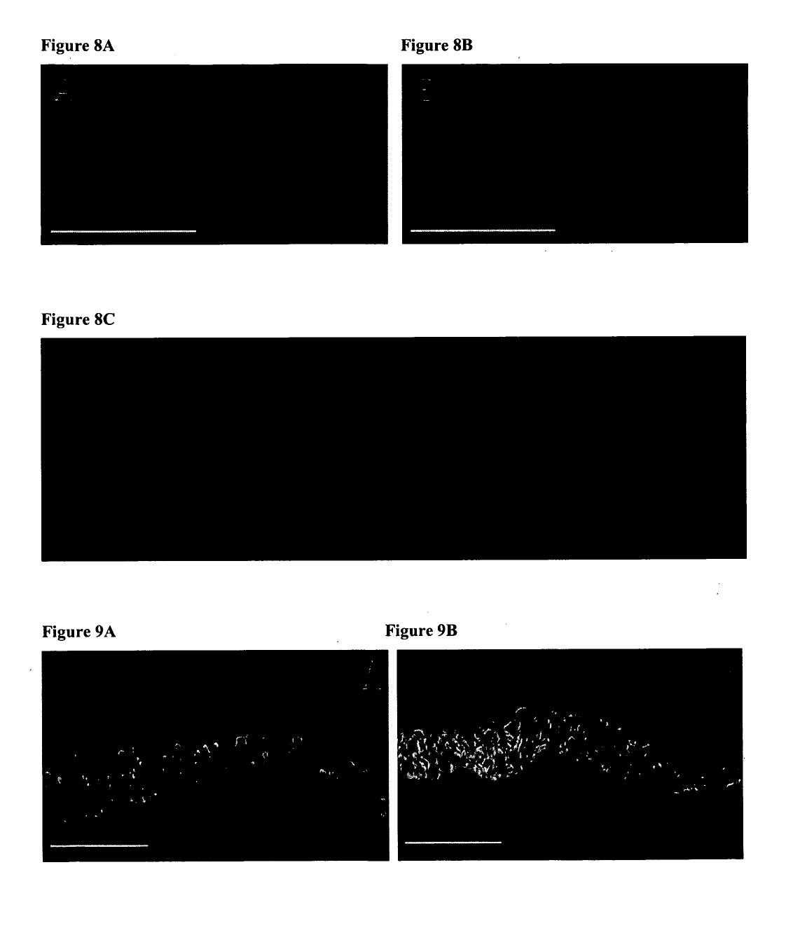

Nuclear (DAPI) staining showed the degree of stratification of corneal limbal cells between cells expanded using the limbal explants (FIG. 8A) and limbal suspension media (FIG. 8B) after 10-14 days in culture. The stratification of cultivated limbal cells was 3-6 layers after 10-14 days in culture. Stratification to a similar level seen by limbal cells grown on dehydrated (plastically compressed) collagen gels (FIG. 8C).

### Example 14

### Immunochemistry

The resultant corneal constructs, following LECs expansion on compressed collagen gel and denuded AM, were examined by immunofluorescence microscopy. Corneal constructs were embedded in OCT (TissueTek) and frozen in liquid nitrogen then cryosectioned. Prior to immunocytochemistry each section (10 μm thick) was blocked using 5% bovine serum albumin (BSA) in 50 mM Tris-buffered saline (TBS; pH 7.2), containing 0.4% Triton X-100 for 60 min at room temperature. Sections were then incubated overnight at 4° C. with primary antibodies against cytokeratin (CK) 3 (1:50; Chemicon, UK) and CK14 (1:100, Chemicon, UK), diluted in 1% BSA in TBS, containing 0.4% Triton X-100. FITC-labelled secondary antibodies (1:50, Sigma, UK) were used at for lhr at room temperature. Sections were co-stained with propidium iodide (Sigma, UK) and observed by fluorescence microscopy (Carl Zeiss Meditec, Germany).

### Example 15

### K14 and K3 Expression within Cultured Limbal Cells

Suspended limbal epithelial cells showed strong K14 expression (marker for undifferentiated cells) within the basal layer cells, which were negative to CK3 (marker for differentiated cells (FIG. 9A) before airlifting. Three to four layer thick basal cells showed a packed cell spatial arrangement, with little intercellular space. The cell nuclear showed high nuclear/cytoplasm ratio. The suprabasal layer cells were more likely flattened with distinct cell boundaries, and these cells were CK14 negative, and also CK3 negative.

The limbal explant cultured cells in same condition also showed positive staining to CK14 (FIG. 9B), and CK3 was also negative or very weakly staining within the explant cultured cells. Different from suspension cultured cells, CK14 positive cells were seen across all of the cell layers (3-4 layers) and even some individual cells on the top-most suprabasal layer. All of these cells showed a large ratio of nuclear/cytoplasm and very closely packed.

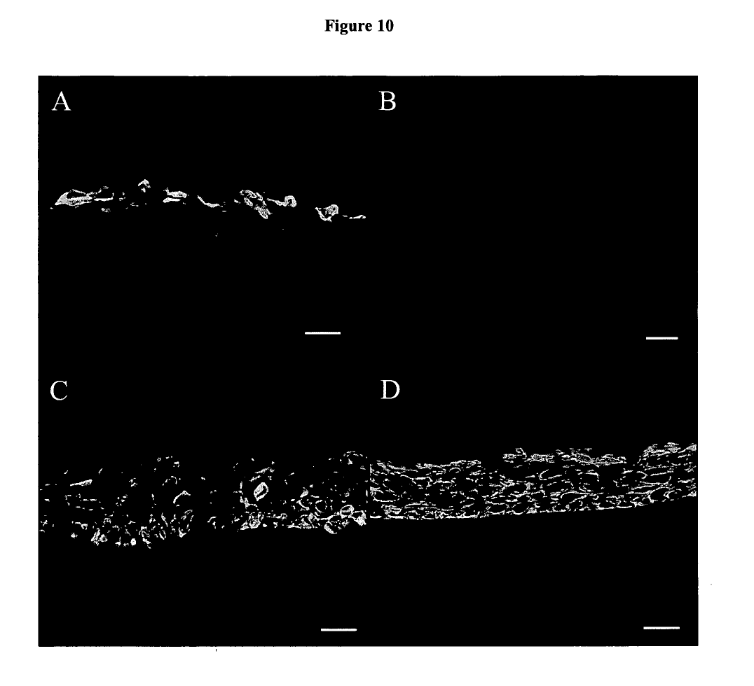

CK3, often used as a specific marker of corneal epithelial cells, was strongly expressed in superficial cell layers of LECs grown on both the compressed collagen gel (FIG. 10A) and denuded AM (FIG. 10B). A further corneal epithelium marker, CK14 (a putative progenitor cell marker), was found to be expressed in all the cell layers of LECs grown on both compressed collagen gel (FIG. 10C) and denuded AM (FIG. 10D).

### Example 16

### Western Blotting

Proteins from LECs grown on compressed collagen scaffold with embedded keratocytes and denuded AM (4 μg total protein for each condition; estimated using the modified Lowry assay), were separated by one-dimensional sodium dodecyl sulphate-polyacrylamide gel electrophoresis (SDS-PAGE) using 10% gels. They were transferred to polyvinylidine difluoride (PVDF) membranes and non-specific binding to membranes was blocked by incubation with 5% (w/v) milk dissolved in 1× Tris-buffered saline-Tween (TBS-T) (20 mM Tris-base, 0.14 M NaCl, 0.1% Tween®-20; pH 7.6). Membranes were incubated with anti-CK3 primary antibody (1 μgml-1) and anti-CK14 primary antibody (1 μgml-1) diluted in 2% (w/v) milk dissolved in 1× TBS-T at 4° C. overnight. Blots were washed for 45 min in 1× TBS-T before incubation with a mouse-conjugated secondary antibody (1:6000 dilution) for 2 h at room temperature. Proteins were detected on X-ray film using an enhanced chemiluminescence system.

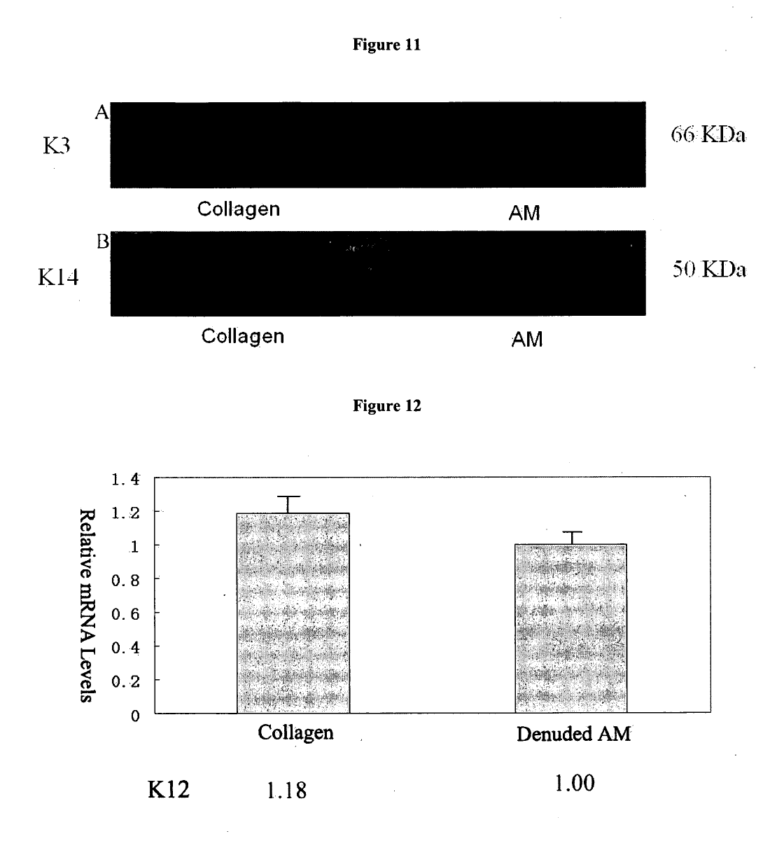

CK3 protein expression was observed in LECs cultured on both scaffolds (compressed collagen and denuded AM), CK3 was more strongly expressed in LECs cultured on compressed collagen substrate than cultured on denuded AM. CK14 protein was also observed in LECs cultured on both scaffolds with no discernible difference in expression levels between the two scaffolds (FIG. 11).

### Example 17

### Real-Time Quantitative PCR

Total RNA was isolated from LECs cultured on both laminin coated compressed collagen scaffold with embedded keratocytes and denuded AM using the TRI reagent (Sigma, Poole, UK), according to the manufacturer's protocol. Total RNA was quantified spectrophotometrically (GE healthcare, UK) and 1 ng RNA was reverse-transribed using RevertAid H Minus First Strand cDNA synthesis Kit (Fermentas, UK), following themanufacturer's protocol. A custom made PerfectProbe assay (PrimerDesign, UK) was used to quantify Keratin 12 (accession number: XM_{—}001255461) gene expression. Each reaction was performed 3 times with a final reaction volume of 20 μl containing 10 μl of 2× qPCR Mastermix (Primerdesign, UK), 1 μl reconsitituted perfect probe primer/probe mix (Primerdesign, UK), 4 μl of PCR-Grade water (Primerdesign, UK) and 5 μl of cDNA (1:10 of original concentration). Non-template controls were also run. Real-time reactions were run on a 96-well plate (Fisher, UK) in the ABI PRISM 7700 Sequence Detector (Applied Biosystem, UK).

A Student's t-test (unpaired) was performed, using Microsoft Excel, to analyse the OD and real-time PCR data. Results are presented as the mean of 3 individual experiments with standard error of mean and P-value≦0.05 was considered significant.

CK12 (like its counterpart CK3) is a marker for differentiated corneal epithelial cells. Using the housekeeping gene, GAPDH, as a control, real time PCR results demonstrated that the CK12 mRNA expression level in LECs expanded upon laminin coated compressed collagen gel embedded with keratocytes (1.18±0.09) was a slightly higher than LECs expanded upon denuded AM (1.00±0.07). This difference was not found to be significant (P>0.05) (FIG. 12).

### Example 18

### Limal Epithelial Outgrowth on Collagen Gels

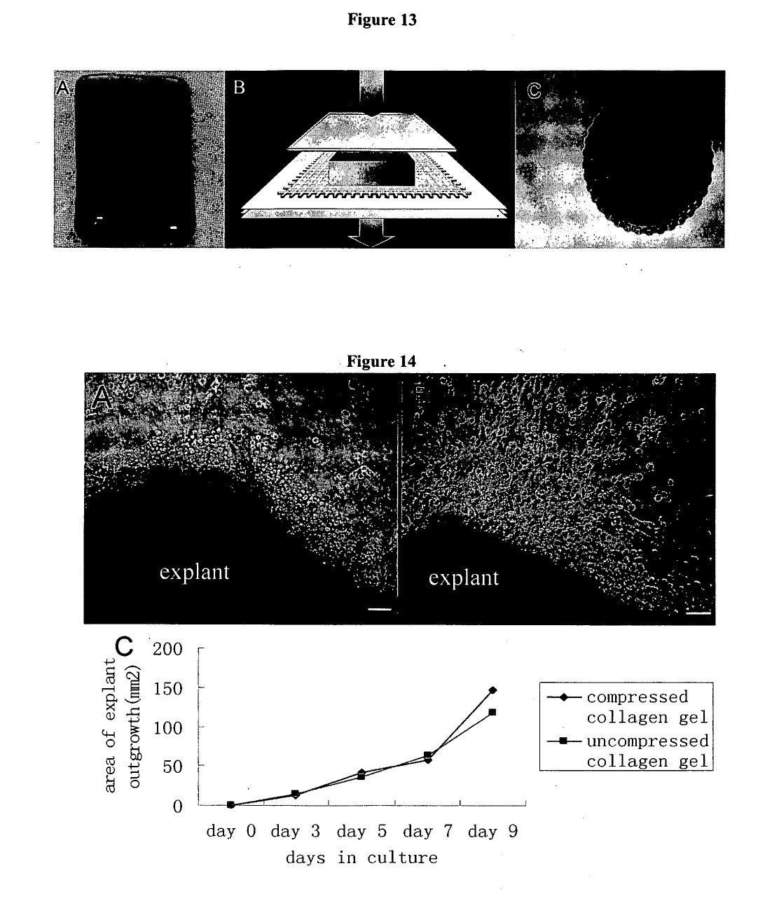

Acellular collagen gels were made as described above. After setting for 30 minutes in the incubator, the collagen gels were well formed (FIG. 13A), the liquid with the compressed gels was expelled by a combination of compression and blotting using layers of nylon mesh and paper sheets (FIG. 13B). The compressed collagen gel was dense, mechanically strong with a high degree of transparency (FIG. 13C).

### Limbal Epithelial Outgrowth on Collagen Gels

Corneal epithelial cells were grown from limbal explants. The remaining intact limbal rims from the previous isolation step were cut into pieces (about 2×2 mm), two pieces with their epithelium side up were directly placed onto the surface of compressed and uncompressed collagen gel and cultured in cell culture medium as described. The area of outgrowth was marked on the top of tissue culture plate while viewing the cells with an inverted microscope. The total area of outgrowth was accurately marked on day 3, 6 and 9, measured and subjected to quantitative analysis.

A Student's t-test (unpaired) was performed to compare LSCs outgrowths on uncompressed and compressed collagen gels using Microsoft Excel. Results are presented as the mean of 3 individual experiments with standard error of mean and P-value≦0.05 was considered significant.

After 3 days, LECs grew out from explants placed on both the uncompressed (FIG. 14A) and compressed (FIG. 14B) collagen gels, and the cells within the outgrowth were observed to be small and regular. The outgrowth areas were marked and measured on day 3, 5, 7 and 9 on uncompressed collagen gel (14.1±0.4, 35.7±1.2, 63.0±2.4, 117.5±5.1; mm²) and compressed collagen gel (12.3±0.4; 41.4±1.3; 57.1±3.2; 147.2±4.8; mm²) respectively. Quantitative analysis of the areas of epithelial outgrowths indicated similar exponential growth on both gel types (P>0.05) (FIG. 14C).

### Ex Vivo Expansion LSCs Suspensions on Collagen Gels

Under sterile conditions; the uncompressed and compressed collagen gels were washed three times with sterilized PBS buffer and then mounted on the bottom of transwell inserts (Corning, UK). A 100 μl suspension of isolated LECs were seeded on to each gel at 10⁶ cells/ml. The cells were cultured in medium as described for 2 weeks then exposed to air by lowering the medium level for 7 days 4. It was important that the medium level was lowered to just meet the surface of the culture, allowing the medium to wet the surface and so the tissue construct remained moist on its apical surface. After 3 weeks of incubation the corneal epithelial construct with multiple layers of cells was ready for examination.

### Electron Microscopy

Compressed and uncompressed collagen gels before and after LECs expansion and the limbal ring after collagenase digestion were examined by scanning electron microscopy (SEM) and transmission electron microscopy (TEM). Specimens were fixed in 2.5% (v/v) glutaraldehyde, washed three times for 10 minutes in PBS, and post-fixed for 2 hours in 1% aqueous osmium tetroxide. Specimens were then washed 3 more times in PBS before being passed through a graded ethanol series (50%, 70%, 90% and 100%). For SEM, specimens were transferred to hexamethyldisilazane for 20 minutes and allowed to air dry. These specimens were then mounted on aluminium stubs and sputter coated with gold before examination using an SEM (FEI Quanta FEG 600, UK). For TEM, the dehydrated specimens were embedded in epoxy resin (Agar 100; Agar Scientific, Ltd., Stansted, UK). Ultrathin (70 nm) sections were collected on copper grids and stained for 1 hr with uranyl acetate and 1% phosphotungstic acid and then for 20 min with Reynolds' lead citrate before examination using a transmission electron microscope (Philips CM20, Holland).

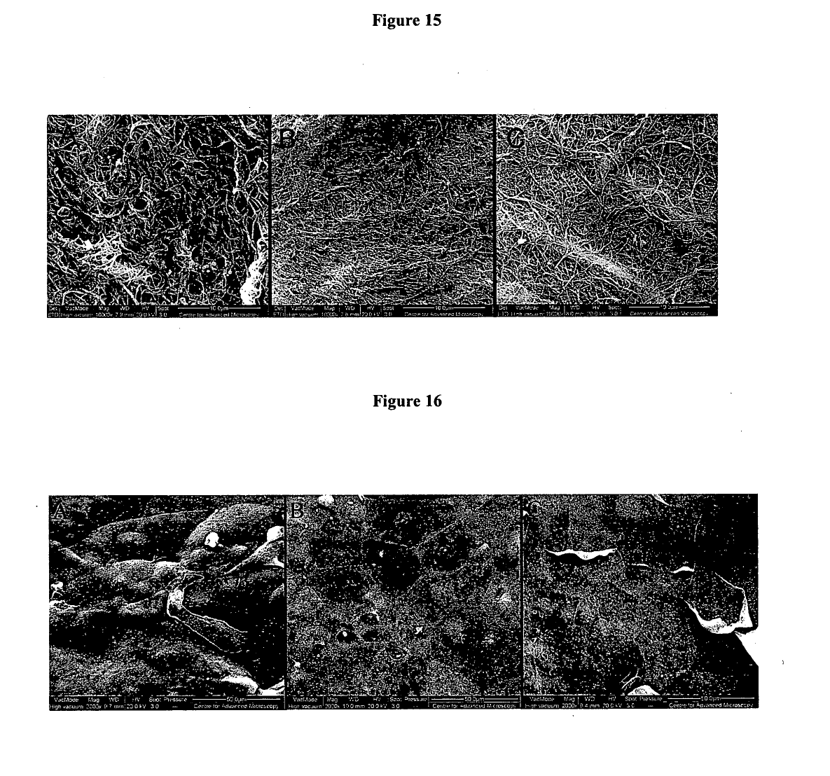

The SEM analyses of the collagen gels showed collagen fibres within the uncompressed gel to be very loosely arranged (FIG. 15A) while within the compressed gel the collagen fibres appeared dense and homogeneous (FIG. 15B) and similar in morphology to the denuded corneal stroma (FIG. 15C). Comparing the relative pore sizes (gaps between collagen fibres), the compressed gel was similar to the denuded stroma with much smaller and more regular pore sizes than the uncompressed gel.

### Scanning Electron Microscopy of LECs Distribution on Different Scaffolds

The LECs were observed to proliferate on both uncompressed and compressed collagen gels. SEM images of cells on uncompressed gels showed that the cells were unevenly distributed and the shape of cells was irregular (FIG. 16A), while the images of cells on compressed gels clearly demonstrated that the cells were more evenly distributed and homogeneous in shape and size (FIG. 16B) the same as that shown by epithelium on normal bovine cornea (FIG. 16C).

### Transmission Electron Microscopy of the Structure of LECs on Different Scaffolds

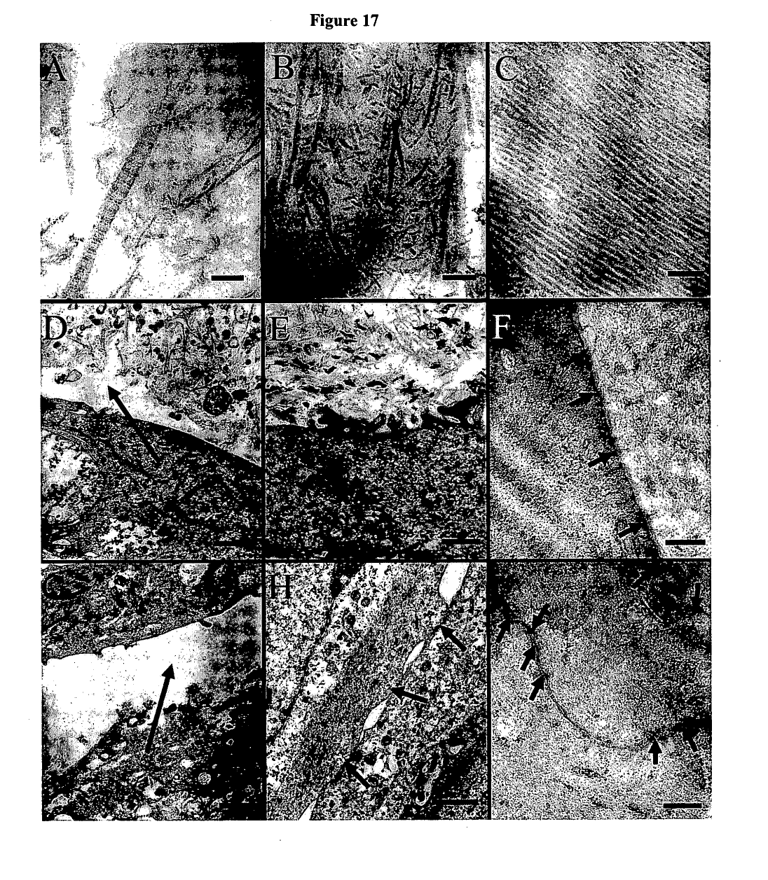

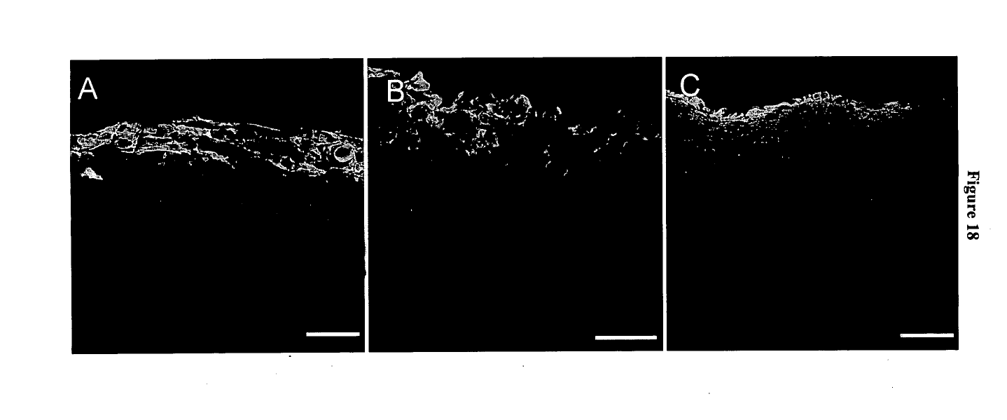

The collagen fibres within the uncompressed collagen gel were loose and of varied diameter (FIG. 17A) while the fibres within the compressed gel were denser, less varied in diameter and more ordered (FIG. 17B). The collagen fibres within compressed gel more closely resembled the normal stromal fibres from bovine cornea (FIG. 17C) than those from the uncompressed scaffold. TEM analyses indicated that the LECs seeded onto uncompressed collagen gels did not form cell matrix attachments nor a substantial basement membrane layer (FIG. 17D). However, LECs expanded upon on compressed gels produced a defined basement membrane layer and evidence of hemidesmosomes formation in the basal cells (FIG. 17E), similar to that shown by normal corneal epithelium (FIG. 17F). Multi-cell layers were formed on the uncompressed collagen gels, but there were large gaps between these cells indicating poor cell-cell attachment (FIG. 17G). On the compressed gel neighbouring cells were attached via desmosome structures (FIG. 17H) again similar to that shown by normal corneal epithelium (FIG. 17I).

### Immunohistochemistry

The resultant corneal constructs, following LECs expansion on collagen gels, were examined by immunofluorescence. Cryosections (10 μm thick) were treated with 5% bovine serum albumin (BSA) in 50 mM Tris-buffered saline (TBS; pH 7.2), containing 0.4% Triton X-100 for 60 min at room temperature. Sections were then incubated overnight at 4° C. with primary antibodies against cytokeratin (CK) 3 (1:50; Chemicon, UK) and CK14 (1:100, Chemidon, UK), diluted in 1%BSA in TBS, containing 0.4% Triton X-100. FITC-labelled secondary antibodies (Sigma, UK) were used. Sections were co-stained with propidium iodide (Sigma, UK) and observed by fluorescence microscopy (Carl Zeiss Meditec, Germany).

The LECs were successfully expanded and stratified upon both forms of collagen scaffold, but were seen to form more cell layers on compressed gels (FIG. 18B) than on uncompressed gels (FIG. 18A), making the compressed group more similar to normal corneal epithelium (FIG. 18C). The propidium iodide (red) stained tissue sections clearly showed inter-nuclei distances to be much larger within the uncompressed group than those in the compressed group. The cell density per mm² on uncompressed, compressed and normal bovine stroma were 0.41, 0.72, 0.81 respectively. CK3 (green), often used as a specific marker of differentiated corneal epithelial cells, was found in the superficial epithelial cells expanded upon uncompressed (FIG. 18A) and compressed collagen gels (FIG. 18B) similar to the normal corneal epithelium (FIG. 18C).

### Example 19

### Toxicity Testing

The ability of the artificial ocular epithelia to measure oculotoxicity accurately is assessed using well-characterised ocular surface toxins and novel nanoparticles on epithelial barrier function, cell viability and morphology.

Test chemicals, selected from the ECETOC data bank, which rank the chemicals for eye irritation potential (ECETOC, Eye Irritation: ECETOC Technical Report. 1998, Reference Chemicals Data Bank, ECETOC, Brussels, Belgium. p. 236) are chosen to represent a range of ocular irritancies (i.e. non, mild, moderate, severe). Liquid sample concentrations use deionised water for dilution in accordance with historical in vivo Draize test records and a positive control of 0.3% Triton X-100. Test materials are applied directly onto the surface of the epithelial cultures (100 μl liquid/suspension or 100 mg solid/powder) for different exposure periods (10, 20, 30 and 60 min). Nanoparticle toxicity is assessed by drop-wise application, of 0.1, 0.5 and 1 nM concentrations of 10-20 nm size gold nanoparticles to the surface of corneal equivalent for 24, 48 and 72 hours. Pegylated gold nanoparticles and gold nanoparticles that have been conjugated to a thermoresponsive block copolymer, poly(N-isopropylacrylamide), forming a corona around each gold nanoparticle are also assessed for cell and tissue toxicity. The induced cytotoxicity (change in cellular proliferation) is quantified by a routinely used colorimetric MTT (3-(4,5-dimethylthiazol-2-yl)-2,5-diphenyltetrazolium bromide) assay (Mosmann, T. J Immunol Methods, 1983. 65(1-2): p. 55-63) and the percentage of viability is calculated. Qualitative measurements of toxicity are achieved by evaluating the frequency and degree of cell surface disruption and the appearance of cellular microplicae and microvilli by scanning electron microscopy (SEM). A previously-developed numerical rating system is used to aid in the categorization of relative damage to corneal epithelia (Burstein, N. Invest. Ophthalmol. Vis. Sci., 1980. 19(3): p. 308-313). Other measures of toxicity include Trypan Blue exclusion cell viability assay and PCR arrays against stress, toxicity and DNA damage associated genes. X-ray microanalysis (EDAX) is also included for nanogold particle localisation and validation.

The model's predictivity is evaluated by investigating the relation of the in vitro stem cell based assay's viabilities with the in vivo Modified Maximum Average Scores (MMAS), a scoring system which quantifies effects on the cornea as reported in the ECETOC data base. Besides the ECETOC report, additional (internet) sources of in vivo data (Toxnet, (http://toxnet.nlm.nih.gov): a cluster of databases on toxicology, hazardous chemicals, and related areas) and results obtained in other alternative test models (e.g. Bovine Corneal Opacity and Permeability test; Slug Mucosal Irritation test; commercial epithelium models), are included in the final validity assessment of the corneal stem cell model.

The amniotic membrane used in the above Examples was obtained from anonymous female donors via Queen Mary's Hospital (UK) and the University of Nottingham (UK). Permission was obtained from Nottingham University. The human corneas were obtained from anonymous human donors via the Royal Berkshire Hospital (UK). Regional Ethical Committee approval was granted for the use of the corneal cells.

### Example 20

### Riboflavin/UV Cross-Linking of Compressed Collagen Gels

In order to improve the mechanical properties of the compressed collagen gels, the collagen fibres in these gels were crosslinked using riboflavin and UV. The basic method is described in Wollensak G. et al. (American Journal of Ophthalmology, Volume 135, Issue 5, May 2003, pages 620-627). Essentially, compressed collagen gels were incubated in 0.1% riboflavin solution (10 mg riboflavin in an 10 mL dextran 20% solution) for 30 mins at room temperature. The irradiation was performed at a 5 cm distance between the collagen gel and a UVA lamp at 365 nm for 30 min. The gels were then washed in PBS to remove any unused riboflavin.

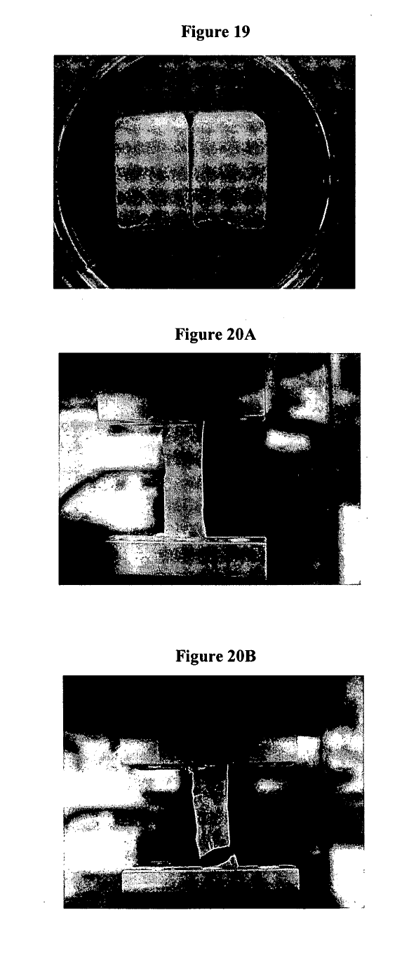

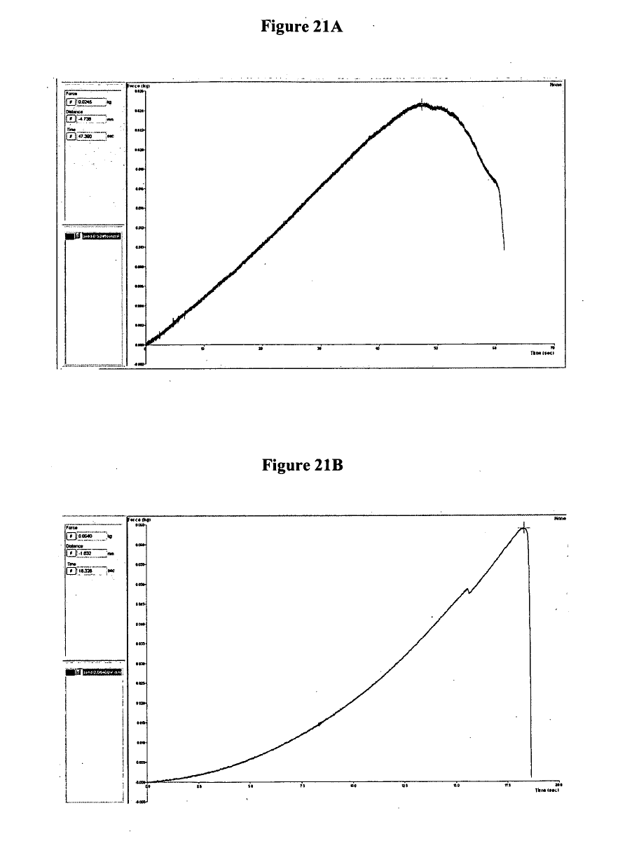

Data on 8 compressed gels is given below. Further information is given in FIGS. 19-21.

| sample | 1 | 2 | 3 | 4 | 5 | 6 | 7 | 8 | mean |
|---|---|---|---|---|---|---|---|---|---|
| Breaking | 0.0294 | 0.0305 | 0.0324 | 0.0383 | 0.0143 | 0.0209 | 0.0218 | 0.0245 | 0.0265 |
| force | | | | | | | | | |
| (untreated) | | | | | | | | | |
| (Kg) | | | | | | | | | |
| Breaking | 0.0504 | 0.0629 | 0.0538 | 0.0640 | 0.0840 | 0.0419 | 0.0512 | 0.0392 | 0.0560 |
| force | | | | | | | | | |
| (riboflavin/ | | | | | | | | | |
| UV treated) | | | | | | | | | |
| (Kg) | | | | | | | | | |

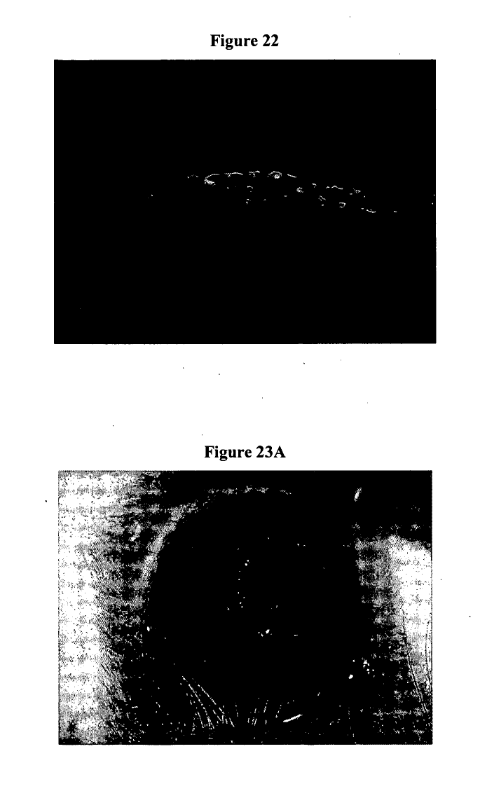

CK3, often used as a specific marker of corneal epithelial cells, was strongly expressed in superficial cell layers of LECs grown on riboflavin/UV treated compressed collagen gel (FIG. 22) similar to that shown by LECs grown on compressed collagen gel (FIG. 10A) and denuded AM (FIG. 10B).

### Example 21

### Clinical Assessment of Transplantation of Compressed Collagen Gels and Riboflavin/UV Treated Compressed Collagen Gels

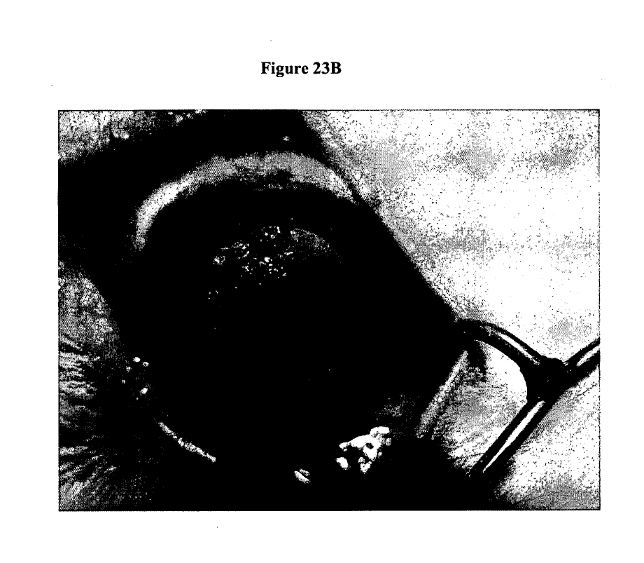

In order to assess the suitability of the compressed collagen gels for use in corneal transplantation a compressed collagen gel (FIG. 23A) and a riboflavin/UV treated collagen gel (FIG. 23B) were sutured onto the wounded rabbit corneas. The rabbit corneas had previously had their ocular surface surgically removed i.e. the corneal epithelial cell layers and part of the underlying stroma (collagen matrix). The riboflavin/UV treated collagen gels, due to their increased mechanical strength (Example 20) could be better held in place resulting in a more successful transplant.

1. An artificial ocular tissue comprising an artificial ocular epithelium and plastically-compacted collagen gel substrate, obtained by or obtainable by a process comprising culturing corneal stem cells or a composition comprising corneal stem cells on a plastically-compacted collagen gel substrate, wherein the cells or the composition are cultured under conditions such as to provide a population of corneal epithelial cells which produce an artificial ocular epithelium on the plastically-compacted collagen gel substrate.

2. A process for producing an artificial ocular epithelium comprising culturing corneal stem cells or a composition comprising corneal stem cells on a plastically-compacted collagen gel substrate, wherein the cells or the composition are cultured under conditions such as to provide a population of corneal epithelial cells which produce an artificial ocular epithelium on the substrate.

3. A process as claimed in claim 2, wherein the artificial ocular epithelium is subsequently isolated from the substrate.

4. A process as claimed in claim 2, wherein the artificial ocular epithelium is subsequently stored in media suitable for the storage and preservation of human tissue, wherein the ocular epithelium is stored with or without the plastically-compacted collagen gel substrate.

5. A process as claimed in claim 2, wherein the corneal stem cells are limbal corneal epithelial stem cells, preferably human limbal corneal epithelial stem cells.

6. A process as claimed in claim 2, wherein the plastically-compacted collagen gel substrate is produced by a process of providing a collagen gel comprising a matrix of collagen fibrils in an interstitial liquid and then plastically-compacting the gel by:
(i) applying a compressing force to one or more of the surfaces or edges of the gel;
(ii) applying a dehydrating force to one or more of the surfaces or edges of the gel;
(iii) stretching the gel in one or two planes; or
(iv) a combination of one or more of (i)-(iii), and optionally subjecting the compacted gel to one or more repetitive cycles of:
(a) applying a uniaxial load along an axis of the gel, and
(b) removing said load.

7. A process as claimed in claim 6, wherein one or more of (i)-(iv) is combined with applying an interstitial-liquid absorbing material to one or more surfaces or edges of the gel.

8. A process as claimed in claim 2, wherein the plastically-compacted collagen gel substrate is I-60 mm in length, preferably 20-40 mm in length and/or 0.5-60 mm in width, preferably 20-40 mm in width.

9. A process as claimed in claim 2, wherein the plastically-compacted collagen gel substrate is 10-1000 μm thick, preferably 20-1000 μm thick.

10. A process as claimed in claim 2, wherein the collagen fibrils in the plastically-compacted collagen gel substrate are 10-100 nm diameter and/or the spacing of the fibrils is 1-200 nm.

11. A process as claimed in claim 2, wherein the collagen content of the plastically-compacted collagen gel substrate is 3-4%.

12. A process as claimed in claim 2, wherein at least one surface of the compacted collagen gel is coated with laminin or one or more laminin domains, and the corneal stem cells or composition are cultured on the laminin/laminin domain surface.

13. A process as claimed in claim 2, wherein the compacted collagen gel comprises stromal progenitor cells, preferably corneal fibroblasts, entrapped within the gel.

14. A process as claimed in claim 2, wherein the collagen in the compacted collagen gel has been cross-linked, preferably using riboflavin and exposure to UV.

15. A process as claimed in claim 2, wherein the plastically-compacted collagen gel is compacted to an extent which prevents ingrowth of the corneal stem cells into the gel.

16. A process as claimed in claim 2, wherein the plastically-compacted collagen gel is flexible and non-rigid.

17. A process as claimed in claim 2, wherein the artificial ocular epithelium is subsequently retained on the substrate, thus forming an artificial ocular tissue.

18. An artificial ocular epithelium obtained by or obtainable by a process as claimed in claim 2.

19. An artificial ocular epithelium comprising a continuous stratified epithelium of 3-7 cell layers expressing both CK3 differentiation marker and CK14 undifferentiation marker with basal membrane components within and beneath the basal cells.

20. An artificial ocular epithelium as claimed in claim 19, wherein hemidesmosomes are present in some or all basal cells and/or some or all neighbouring epithelial cells are attached to each other via desmosome structures.

21. An artificial ocular tissue obtained by or obtainable by a process as claimed in claim 17.

22. An artificial ocular tissue comprising:
(i) an artificial ocular epithelium as claimed in claim 19; and
(ii) a plastically-compacted collagen gel substrate.

23. A method of assessing the effect of a test compound on an artificial ocular epithelium, comprising the steps:
(a) providing an artificial ocular epithelium as claimed in claim 19;
(b) contacting the artificial ocular epithelium with an amount of the test compound; and
(c) assessing the effect of the compound on the artificial ocular epithelium.

24. -26. (canceled)

27. A method of treating an ocular injury comprising:
(a) providing an artificial ocular epithelium as claimed in claim 19;
(b) contacting the ocular injury with said artificial ocular epithelium; and optionally
(c) securing the said artificial ocular epithelium at the site of the ocular injury.

28.-29. (canceled)

30. A method as claimed in claim 27, wherein the ocular injury is one related to an insufficient stromal microenvironment to support stem cell function, such as aniridia, keratitis, neurotrophic keratopathy and chronic limbitis; or related to external factors that destroy limbal stem cells, such as chemical or thermal injuries, Stevens-Johnson syndrome, ocular cicatricial pemphigoid, contact lens wear, or extensive microbial infection.

31. A method of replacing a cornea in a mammalian subject comprising:
(a) providing an artificial ocular epithelium as claimed in claim 19;
(b) replacing the cornea of the mammalian subject with said artificial ocular epithelium.

32.-45. (canceled)

46. A method according to claim 23, wherein the artificial ocular epithelium is present in artificial ocular tissue which further comprises a plastically-compacted collagen gel substrate.

47. A method according to claim 27, wherein the artificial ocular epithelium is present in artificial ocular tissue which further comprises a plastically-compacted collagen gel substrate.

48. A method according to claim 31, wherein the artificial ocular epithelium is present in artificial ocular tissue which further comprises a plastically-compacted collagen gel substrate.
